(19) United States
(12) Patent Application Publication
(10) Pub. No.: US 2008/0039398 A1
(43) Pub. Date: Feb. 14, 2008

(54) Formulations and methods for treating dry eye

(76) Inventors: George W. Ousler III, North Andover, MA (US); Matthew Jonathan Chapin, Amesbury, MA (US); Mark B. Abelson, Andover, MA (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 11/890,030

(22) Filed: Aug. 2, 2007

Related U.S. Application Data
(63) Continuation-in-part of application No. 11/820,461, filed on Jun. 18, 2007.
Application No. 11/820,461 is a continuation-in-part of application No. 11/807,147, filed on May 24, 2007.
Application No. 11/807,147 is a continuation-in-part of application No. 11/698,778, filed on Jan. 25, 2007.
(60) Provisional application No. 60/761,945, filed on Jan. 25, 2006.

(51) Int. Cl. A61K 38/00; A61K 31/01; A61K 31/045; A61K 31/21; A61P 27/04; A61K 31/40; A61K 31/715; A61K 31/717

(52) U.S. Cl. 514/12; 514/413; 514/57; 514/648; 514/738; 514/762

(57) ABSTRACT

The present invention provides compositions for treating and/or preventing signs and symptoms associated with dry eye and/or ocular irritation, and methods of use thereof. Such compositions are provided in novel ophthalmic formulations that are comfortable upon instillation in the eye.

### RELATED APPLICATIONS

This application is a continuation-in-part of U.S. Ser. No. 11/820,461, filed Jun. 18, 2007, which is a continuation-in-part of U.S. Ser. No. 11/807,147, filed May 24, 2007, which is a continuation-in-part of U.S. Ser. No. 11/698,778, filed Jan. 25, 2007, which claims the benefit of U.S. Provisional Application No. 60/761,945, filed Jan. 25, 2006, the contents of which are each hereby incorporated by reference in their entirety.

### FIELD OF THE INVENTION

The invention relates generally to compositions for the treatment of ocular disorders, and more particularly to compositions comprising a tear substitute, or one or more components thereof, and a second agent for the treatment of acute or chronic dry eye disease. The invention further relates to materials and methods for the administration of compositions comprising a tear substitute, or one or more components thereof and a second agent.

### BACKGROUND OF THE INVENTION

Dry eye disease is an ocular disease affecting approximately 10-20% of the population. This disease progressively affects larger percentages of the population as it ages, with the majority of these patients being women. In addition, almost everyone experiences ocular irritation, or the symptoms and/or signs of dry eye as a condition, from time to time under certain circumstances, such as prolonged visual tasking (e.g. working on a computer), being in a dry environment, using medications that result in ocular drying, etc.

In individuals suffering from dry eye, the protective layer of tears that normally protects the ocular surface is compromised, a result of insufficient or unhealthy production of one or more tear components. This can lead to exposure of the surface of the eye, ultimately promoting desiccation and damage of surface cells. Signs and symptoms of dry eye include but are not limited to keratitis, conjunctival and corneal staining, redness, blurry vision, decreased tear film break-up time, decreased tear production, tear volume, and tear flow, increased conjunctival redness, excess debris in the tear film, ocular dryness, ocular grittiness, ocular burning, foreign body sensation in the eye, excess tearing, photophobia, ocular stinging, refractive impairment, ocular sensitivity, and ocular irritation. Patients may experience one or more of these symptoms. The excess tearing response may seem counterintuitive, but it is a natural reflex response to the irritation and foreign body sensation caused by the dry eye. Some patients may also experience ocular itching due to a combination of ocular allergy and dry eye symptoms.

There are many possible variables that can influence a patient's signs or symptoms of dry eye including levels of circulating hormones, various autoimmune diseases (e.g. Sjögren's syndrome and systemic lupus erythematosus), ocular surgeries including PRK or LASIK, many medications, environmental conditions, visual tasking such as computer use, ocular fatigue, contact lens wear, and mechanical influences such as corneal sensitivity, partial lid closure, surface irregularities (e.g. pterygium), and lid irregularities (e.g. ptosis, entropion/ectropion, pinguecula). Environments with low humidity, e.g., those that cause dehydration, can exacerbate or cause dry eye symptoms, such as sitting in a car with the defroster on or living in a dry climate zone. In addition, visual tasking can exacerbate symptoms. Tasks that can greatly influence symptoms include watching TV or using a computer for long periods of time where the blink rate is decreased.

There are a number of products for the treatment of dry eye commercially available. However, such products provide only temporary relief of acute symptoms, are suitable for short term use only, and/or cause ocular discomfort upon installation in the eye. For example, artificial tears and ointments may provide temporary relief of dry eye, but do little to arrest or reverse any damaging conditions. For more severe cases of dry eye, in which the cornea is inflamed, anti-inflammatory agents are sometimes prescribed. Topical corticosteroids (in eye drops) are safe for short-term use to combat inflammation, but can cause side effects, including but not limited to decreased wound healing, cataract, and in some cases, increased risk of elevated intra-ocular pressure in patients, when used for a long time. Likewise, non-steroidal anti-inflammatory drugs (NSAIDs) in their current ophthalmic dosage forms are approved for short term use only, e.g., inflammation and pain associated with post-ocular-surgery, and may result in corneal damage in patients predisposed to such conditions, delayed wound healing after repeated dosing, or ocular discomfort. (see e.g., Congdon et al., J Cataract Refract Surg. 2001 April; 27(4):622-31; Flach A., Tr Am Ophthal Soc 2001; 99:205-212).

Commercial cyclosporin-A (Restasis®-Allergan) is the first approved therapeutic agent for the treatment of dry eye, and is suitable for long term use. However, the primary side effect cited on the package insert is ocular burning and stinging upon instillation, and Restasis® was only shown to be effective in only 17% of patients. To improve patient discomfort during the induction phase of cyclosporin therapy, clinicians may prescribe topical corticosteroids or NSAIDs (in eye drop form) in conjunction with cyclosporin-A (see e.g., Schechter B., J Ocul Pharmacol Ther. 2006 April; 22(2):150-4). However, such agents in their current ophthalmic dosage forms should only be used during the initiation of cyclosporin treatment, due to the potential adverse effects of damage to the cornea, delayed wound healing, and discomfort associated with such dosage forms. As such, there exists a need for an ocular therapy for the treatment of acute or chronic dry eye disease which is comfortable upon instillation in the eye, and at a safe dose particularly suitable for long term use. The present invention meets this need and other needs.

### SUMMARY OF THE INVENTION

The present invention provides ophthalmic formulations suitable for the treatment of acute or chronic dry eye disease which contain a combination of ingredients capable of acting synergistically to relieve ocular discomfort and prolong the integrity of the tear film. In particular, the formulations described herein provide an NSAID suitable for ophthalmic use in a comfortable ophthalmic formulation when instilled in the eye. Specifically provided are ophthalmic formulations comprising one or more components of a tear substitute and a low dose amount of NSAID effective to treat and/or prevent signs and symptoms associated with dry eye disease, suitable for intermittent and/or repeated long term use for the treatment of chronic dry eye disease.

In some embodiments, the ophthalmic formulations of the invention comprise a low dose amount of an NSAID selected from the group consisting of: ketorolac tromethamine (also referred to herein as ketorolac), indomethacin, flurbiprofen sodium, nepafenac, bromfenac, suprofen and diclofenac. Other suitable NSAIDs may be used.

In one embodiment of the invention, the low dose amount of NSAID in the ophthalmic formulation of the invention is about 0.10% to about 0.30%, more preferably about 0.15% to about 0.30%, even more preferably about 0.15% to about 0.26% ketorolac tromethamine. In another embodiment the low dose amount of NSAID is about 0.01% to about 0.10%, preferably about 0.03% to about 0.08%, more preferably about 0.040% to about 0.065% indomethacin. In another embodiment, the low dose amount of NSAID is about 0.009 to about 0.024% flurbiprofen sodium. In another embodiment, low dose amount of NSAID is about 0.03% to about 0.08% nepafenac. In yet another embodiment, low dose amount of NSAID is about 0.027% to about 0.072% bromfenac. In another embodiment, low dose amount of NSAID is about 0.3% to about 0.8% suprofen. In yet another embodiment, the low dose amount of NSAID is about 0.01% to about 0.08% diclofenac.

In some embodiments, the ophthalmic formulations of the invention comprise a tear substitute comprising an active ingredient, which may include without limitation: a polyol, a dextran, a water soluble protein, a carbomer, a gum, a cellulose derivative, or cominbations thereof. Other suitable tear substitute components known in the art may be used in the formulations of the invention. Suitable cellulose derivatives for use in the ophthalmic formulations of the invention include, without limitation, hydroxypropylmethyl cellulose (HPMC), carboxymethyl cellulose (CMC) sodium, hydroxypropyl cellulose, hydroxyethyl cellulose, methyl cellulose, or combinations thereof. In preferred embodiments, the cellulose derivative is hydroxypropylmethyl cellulose and/or carboxymethyl cellulose sodium.

In a preferred embodiment, the viscosity of the tear substitute, or one or more components thereof, is in a range which optimizes efficacy of supporting the tear film while minimizing blurring, lid caking, etc. Preferably, the viscosity of the tear substitute, or one or more components thereof, ranges from about 30-150 centipoise (cpi), preferably about 30-130 cpi, more preferably about 50-120 cpi, even more preferably about 70-120 cpi. In some embodiments, the viscosity of the tear substitute, or one or more components thereof, ranges from about 50-90 cpi.

In some embodiments, the invention features an ophthalmic formulation comprising a combination of a tear substitute component having a viscosity of about 50 cpi, and a low dose amount of an NSAID, useful for the treatment and/or prevention of the signs and/or symptoms of dry eye disease. In one preferred embodiment, the ophthalmic formulation of the invention comprises a combination of 0.2% ketorolac tromethamine, and a tear substitute, or one or more components thereof, having a viscosity of about 50 cpi. In another preferred embodiment, the ophthalmic formulation of the invention comprises a combination of 0.25% ketorolac tromethamine and a tear substitute, or one or more components there, having a viscosity of about 50 cpi. In another preferred embodiment, the ophthalmic formulation of the invention comprises a combination of 0.05% nepafenac, and a tear substitute, or one or more components thereof, having a viscosity of about 50 cpi. In yet another preferred embodiment, the ophthalmic formulation of the invention comprises a combination of 0.045% bromfenac, and a tear substitute, or one or more components thereof, having a viscosity of about 50 cpi.

In other embodiments, the invention features an ophthalmic formulation comprising a combination of a tear substitute component having a viscosity of about 70 cpi, and a low dose amount of an NSAID, useful for the treatment and/or prevention of the signs and/or symptoms of dry eye disease. In one preferred embodiment, the ophthalmic formulation of the invention comprises a combination of 0.2% ketorolac tromethamine, and a tear substitute, or one or more components thereof, having a viscosity of about 70 cpi. In another preferred embodiment, the invention features an ophthalmic formulation comprising a combination of 0.25% ketorolac tromethamine, and a tear substitute, or one or more components thereof, having a viscosity of about 70 cpi. In another preferred embodiment, the ophthalmic formulation of the invention comprises a combination of 0.05% nepafenac, and a tear substitute, or one or more components thereof, having a viscosity of about 70 cpi. In yet another preferred embodiment, the ophthalmic formulation of the invention comprises a combination of 0.045% bromfenac, and a tear substitute, or one or more components thereof, having a viscosity of about 70 cpi.

In still other embodiments, the invention features an ophthalmic formulation comprising a combination of a tear substitute component having a viscosity of about 85 cpi, and a low dose amount of an NSAID, useful for the treatment and/or prevention of the signs and/or symptoms of dry eye disease. In one preferred embodiment, the ophthalmic formulation of the invention comprises a combination of 0.2% ketorolac tromethamine, and a tear substitute, or one or more components thereof, having a viscosity of about 85 cpi. In another preferred embodiment, the invention features an ophthalmic formulation comprising a combination of 0.25% ketorolac tromethamine, and a tear substitute, or one or more components thereof, having a viscosity of about 85 cpi. In another preferred embodiment, the ophthalmic formulation of the invention comprises a combination of 0.05% nepafenac, and a tear substitute, or one or more components thereof, having a viscosity of about 85 cpi. In yet another preferred embodiment, the ophthalmic formulation of the invention comprises a combination of 0.045% bromfenac, and a tear substitute, or one or more components thereof, having a viscosity of about 85 cpi.

In yet another embodiment, the invention features an ophthalmic formulation comprising a combination of a tear substitute component having a viscosity of about 100 cpi, and a low dose amount of an NSAID, useful for the treatment and/or prevention of the signs and/or symptoms of dry eye disease. In one preferred embodiment, the ophthalmic formulation of the invention comprises a combination of 0.2% ketorolac tromethamine, and a tear substitute, or one or more components thereof, having a viscosity of about 100 cpi. In another preferred embodiment, the invention features an ophthalmic formulation comprising a combination of 0.25% ketorolac tromethamine, and a tear substitute, or one or more components thereof, having a viscosity of about 100 cpi. In another preferred embodiment, the ophthalmic formulation of the invention comprises a combination of 0.05% nepafenac, and a tear substitute, or one or more components thereof, having a viscosity of about 100 cpi. In yet another preferred embodiment, the ophthalmic formulation of the invention comprises a combination of 0.045% bromfenac, and a tear substitute, or one or more components thereof, having a viscosity of about 100 cpi.

In another embodiment, the invention features an ophthalmic formulation comprising a combination of a tear substitute component having a viscosity of about 120 cpi, and a low dose amount of an NSAID, useful for the treatment and/or prevention of the signs and/or symptoms of dry eye disease. In one preferred embodiment, the ophthalmic formulation of the invention comprises a combination of 0.2% ketorolac tromethamine, and a tear substitute, or one or more components thereof, having a viscosity of about 120 cpi. In another preferred embodiment, the invention features an ophthalmic formulation comprising a combination of 0.25% ketorolac tromethamine, and a tear substitute, or one or more components thereof, having a viscosity of about 120 cpi. In another preferred embodiment, the ophthalmic formulation of the invention comprises a combination of 0.05% nepafenac, and a tear substitute, or one or more components thereof, having a viscosity of about 120 cpi. In yet another preferred embodiment, the ophthalmic formulation of the invention comprises a combination of 0.045% bromfenac, and a tear substitute, or one or more components thereof, having a viscosity of about 120 cpi.

Also featured are methods of improving, relieving, treating, preventing, or otherwise decreasing ocular discomfort, and methods for increasing tear film break-up time and/or the ocular protection index (as described further herein) for the treatment and prevention of the signs and symptoms associated with dry eye and/or eye irritation by administration of the formulations of the invention. In one embodiment, the method for treating dry eye and/or eye irritation comprises the steps of a) determining a first measurement of the tear film break-up time (TFBUT) and/or ocular protection index (OPI), and/or non-invasive tear film break-up time and/or ocular discomfort in a subject; (b) administering an ophthalmic formulation of the invention to the subject; (c) determining a second measurement of the TFBUT and/or OPI and/or non-invasive tear film break-up time and/or ocular discomfort in a subject; wherein an increase in the second measurement of TFBUT and/or OPI and/or non-invasive tear film break-up time and/or ocular discomfort as compared to the first measurement indicates the ophthalmic formulation is efficacious in treating the subject.

An additional feature is the use of an NSAID in the manufacture of a comfortable ophthalmic formulation for instillation into the eye, wherein said formulation comprises a low dose amount of an NSAID suitable for ophthalmic use and one or more tear substitute components. Also featured are ophthalmic formulations that are comfortable upon instillation in the eye comprising a low dose amount of an NSAID suitable for ophthalmic use and one or more tear substitute components, wherein the tear substitute component is selected from the group consisting of hydroxypropylmethyl cellulose (HPMC) and carboxymethyl cellulose (CMC) sodium, or a combination thereof.

Further, featured are kits for the shipping, storage or use of the formulations, as well the practice of the methods. Other features and advantages of the invention will become apparent from the following detailed description and claims.

### BRIEF DESCRIPTION OF THE DRAWINGS

FIG. 1 is a graph depicting a comparison of the efficacy of Acular® (ketorolac tromethamine 0.5%, also referred to herein as “Acular” or “ketorolac”) and Refresh® artificial tears on reducing ocular discomfort in patients exposed to a controlled adverse environment (CAE).

FIG. 2 is a graph depicting the efficacy of a combination of Acular® (ketorolac tromethamine 0.5%) and a hydroxypropylmethyl cellulose based artificial tear (1:1 dilution, final concentration ketorolac tromethanine 0.25%), as compared to the artificial tear alone, on reducing ocular discomfort in patients exposed to a CAE, over a 60 minute timecourse.

FIG. 3 is a graph depicting the efficacy of a combination of Acular LS® (ketorolac tromethamine 0.4%) and an artificial tear having a viscosity of about 50 cpi (1:1 dilution, final concentration ketorolac tromethamine 0.2%), as compared to the artificial tear alone, on reducing ocular discomfort in patients exposed to a CAE, over a 60 minute timecourse.

FIG. 4 is a graph depicting the efficacy of a combination of Nevanac® (nepafenac 0.1%) and an artificial tear having a viscosity of about 50 cpi (1:1 dilution, final concentration nepafenac 0.05%), as compared to the artificial tear alone, on reducing ocular discomfort in patients exposed to a CAE, over a 60 minute timecourse.

FIG. 5 is a graph depicting the efficacy of a combination of Xibrom® (bromfenac 0.09%) and an artificial tear having a viscosity of about 50 cpi (1:1 dilution, final concentration bromfenac 0.045%), as compared to the artificial tear alone, on reducing ocular discomfort in patients exposed to a CAE, over a 60 minute timecourse.

FIG. 6 is a graph depicting the efficacy of a combination of Voltaren® (diclofenac 0.1%) and an artificial tear having a viscosity of about 50 cpi (1:1 dilution, final concentration diclofenac 0.05%), as compared to the artificial tear alone, on reducing ocular discomfort in patients exposed to a CAE, over a 60 minute timecourse.

FIG. 7 is a graph depicting the efficacy of an ophthalmic formulation comprising ketorolac tromethamine 0.25% and an artificial tear having a viscosity of about 70 cpi, as compared to the artificial tear alone, on reducing ocular discomfort in reducing ocular discomfort in patients exposed to a CAE, over a 60 minute timecourse.

FIG. 8 is a graph depicting the efficacy of an ophthalmic formulation comprising ketorolac tromethamine 0.125% and an artificial tear having a viscosity of about 70 cpi, as compared to the artificial tear alone, on reducing ocular discomfort in patients exposed to a CAE, over a 60 minute timecourse.

FIG. 9 is a graph depicting the efficacy of an ophthalmic formulation comprising ketorolac tromethamine 0.0.06% and an artificial tear having a viscosity of about 70 cpi, as compared to the artificial tear alone, on reducing ocular discomfort in patients exposed to a CAE, over a 60 minute timecourse.

FIG. 10 is a graph depicting a comparison of the efficacy of three different ophthalmic formulations; 1) 0.25% ketorolac tromethamine and a hydroxymethylpropyl cellulose based artificial tear having a viscosity of approximately 50 cpi (depicted as C-2); 2) 0.25% ketorolac tromethamine and a hydroxymethylpropyl cellulose based artificial tear having a viscosity of approximately 50 cpi (depicted as D-2); and 0.5% ketorolac (Acular®) diluted 1:1 with a carboxymethyl cellulose based artificial tear having a viscosity of approximately 80 cpi (final concentration of 0.25% ketorolac, depicted as Liquigel); on increasing tear film break-up time (TFBUT), using procedures for measuring TFBUT over 60 minutes following dosing.

FIG. 11 is a graph depicting a comparison of the efficacy of three different ophthalmic formulations; 1) 0.25% ketorolac and a hydroxymethylpropyl cellulose based artificial tear having a viscosity of approximately 50 cpi (depicted as C-2); 2) 0.25% ketorolac and a hydroxymethylpropyl cellulose based artificial tear having a viscosity of approximately 50 cpi (depicted as D-2); and 0.5% ketorolac (Acular®) diluted 1:1 with a carboxymethyl cellulose based artificial tear having a viscosity of approximately 80 cpi (final concentration of 0.25% ketorolac, depicted as Liquigel); on increasing the Ocular Protection Index (OPI) over 60 minutes following dosing.

FIG. 12 is a graph depicting the efficacy of a combined formulation of Nevanac® (nepafenac 0.1%) and Systane® artificial tear solution (1:1 dilution, final concentration nepafenac 0.1%) (depicted as NEVANAC) as compared to Systane® artificial tear alone (depicted as SYSTANE) on reducing ocular discomfort in patients exposed to a to a CAE, over a 60 minute timecourse.

### DETAILED DESCRIPTION OF THE INVENTION

For convenience, before further description of the present invention, certain terms employed in the specification, examples, and appended claims are collected here. These definitions should be read in light of the remainder of the disclosure and understood as by a person of skill in the art.

The term “acute” as used herein denotes a condition having a rapid onset, and symptoms that are severe but short in duration.

The term “analgesic” as used herein denotes a compound/formulation for the management of intermittent and/or chronic physical discomfort, suitable for long term use.

The term “anesthetic” or “anesthesia” as used herein denotes a compound/formulation for the management of acute physical pain, suitable for short term, temporary use, which has an effect that produces numbing or decreased sensitivity in the body part/organ to which the compound/formulation is administered (e.g., decreased corneal sensitivity of the eye).

The term “aqueous” typically denotes an aqueous composition wherein the carrier is to an extent of >50%, more preferably >75% and in particular 290% by weight water.

The term “chronic” as defined herein is meant a persistent, lasting condition, or one marked by frequent recurrence, preferably a condition that persists/recurs for greater than 3 months, more preferably greater than 6 months, more preferably greater than 12 months, and even more preferably greater than 24 months.

The term “comfortable” as used herein refers to a sensation of physical well being or relief, in contrast to the physical sensation of pain, burning, stinging, itching, irritation, or other symptoms associated with physical discomfort.

The term “comfortable ophthalmic formulation” as used herein refers to an ophthalmic formulation which provides physical relief from symptoms associated with dry eye disease and/or ocular discomfort, and only causes an acceptable level of pain, burning, stinging, itching, irritation, or other symptoms associated with ocular discomfort, when instilled in the eye, which are less than those seen with dosing with current concentrations on the market.

The term “dry eye” as used herein, refers to inadequate tear production and/or abnormal tear composition. Causes of dry eye disease as defined herein include but are not limited to the following: idiopathic, congenital alacrima, xerophthalmia, lacrimal gland ablation, and sensory denervation; collagen vascular diseases, including rheumatoid arthritis, Wegener's granulomatosis, and systemic lupus erythematosus; Sjögren's syndrome and autoimmune diseases associated with Sjögren's syndrome; abnormalities of the lipid tear layer caused by blepharitis or rosacea; abnormalities of the mucin tear layer caused by vitamin A deficiency; trachoma, diphtheric keratoconjunctivitis; mucocutaneous disorders; aging; menopause; and diabetes. Dry eye signs and/or symptoms as defined herein may also be provoked by other circumstances, including but not limited to the following: prolonged visual tasking; working on a computer; being in a dry environment; ocular irritation; contact lenses, LASIK and other refractive surgeries; fatigue; and medications such as isotretinoin, sedatives, diuretics, tricyclic antidepressants, antihypertensives, oral contraceptives, antihistamines, nasal decongestants, beta-blockers, phenothiazines, atropine, and pain relieving opiates such as morphine.

The phrase “effective amount” is an art-recognized term, and refers to an amount of an agent that, when incorporated into a pharmaceutical composition of the present invention, produces some desired effect at a reasonable benefit/risk ratio applicable to any medical treatment. In certain embodiments, the term refers to that amount necessary or sufficient to eliminate, reduce or maintain (e.g., prevent the spread of) a sign and/or symptom of dry eye and/or eye irritation, or prevent or treat dry eye and/or eye irritation. The effective amount may vary depending on such factors as the disease or condition being treated, the particular composition being administered, or the severity of the disease or condition. One of skill in the art may empirically determine the effective amount of a particular agent without necessitating undue experimentation.

As used herein, the term “NSAID” means an opthalmologically acceptable nonsteroidal anti-inflammatory drug or a pharmaceutically acceptable salt thereof. The term “low dose NSAID” means an amount of an opthalmologically acceptable nonsteroidal anti-inflammatory drug or a pharmaceutically acceptable salt thereof, which reduces ocular discomfort without producing anesthesia, and which would be expected to have reduced adverse effects associated with current FDA approved formulations of NSAIDs marketed for the treatment of acute ocular inflammation and pain, including without limitation, corneal damage, delayed wound healing, and ocular discomfort.

A “patient,” “subject,” or “host” to be treated by the subject method refers to either a human or non-human animal, such as a primate, mammal, and vertebrate

The phrase “pharmaceutically acceptable” is art-recognized and refers to compositions, polymers and other materials and/or salts thereof and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” is art-recognized, and refers to, for example, pharmaceutically acceptable materials, compositions or vehicles, such as a liquid or solid filler, diluent, excipient, solvent or encapsulating material, involved in carrying or transporting any supplement or composition, or component thereof, from one organ, or portion of the body, to another organ, or portion of the body, or to deliver an agent to the surface of the eye. Each carrier must be “acceptable” in the sense of being compatible with the other ingredients of the composition and not injurious to the patient. In certain embodiments, a pharmaceutically acceptable carrier is non-pyrogenic. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, hydroxypropylmethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, sunflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; (21) gums such as HP-guar; (22) polymers; and (23) other non-toxic compatible substances employed in pharmaceutical formulations.

The term “pharmaceutically acceptable salts” is art-recognized, and refers to relatively non-toxic, inorganic and organic acid addition salts of compositions of the present invention or any components thereof, including without limitation, therapeutic agents, excipients, other materials and the like. Examples of pharmaceutically acceptable salts include those derived from mineral acids, such as hydrochloric acid and sulfuric acid, and those derived from organic acids, such as ethanesulfonic acid, benzenesulfonic acid, ptoluenesulfonic acid, and the like. Examples of suitable inorganic bases for the formation of salts include the hydroxides, carbonates, and bicarbonates of ammonia, sodium, lithium, potassium, calcium, magnesium, aluminum, zinc and the like. Salts may also be formed with suitable organic bases, including those that are non-toxic and strong enough to form such salts. For purposes of illustration, the class of such organic bases may include mono-, di-, and trialkylamines, such as methylamine, dimethylamine, and triethylamine; mono-, di- or trihydroxyalkylamines such as mono-, di-, and triethanolamine; amino acids, such as arginine and lysine; guanidine; N-methylglucosamine; N-methylglucamine; L-glutamine; N-methylpiperazine; morpholine; ethylenediamine; N-benzylphenethylamine; (trihydroxymethyl)aminoethane; tromethamine, and the like. See, e.g., J. Pharm. Sci., 66: 1-19 (1977).

The term “preventing,” when used in relation to a condition, such as dry eye and/or eye irritation, is art-recognized, and refers to administration of a composition which reduces the frequency of, or delays the onset of, signs and/or symptoms of a medical condition in a subject relative to a subject which does not receive the composition.

As used herein, the terms “tear substitute” and “artificial tear” may be used interchangeably, and each refers to one or more molecules or compositions, which lubricate, “wet,” approximate the consistency of endogenous tears, aid in natural tear build up, or otherwise provide temporary relief of dry eye signs and/or symptoms and conditions upon ocular administration, including without limitation a polymer (e.g., a cellulosic polymer), an ocular surface protectant, a demulcent, or other component found on the FDA monograph for tear substitutes. The term “tear substitute component” refers to one or more components thereof.

The term “treating” is an art-recognized term which refers to reducing or ameliorating at least one sign and/or symptom of any condition or disease.

1. Pharmaceutical Compositions

The invention features novel pharmaceutical compositions comprising an effective amount of an NSAID and one or more tear substitute components in a pharmaceutically acceptable carrier. The NSAID component provides relief of or prevention of ocular discomfort, and the one or more tear substitute components provide ocular surface protection via enhancement of the tear film (as evident by increased tear film break up time). An effective amount of the formulations may be used to treat and/or prevent signs and symptoms associated with dry eye and/or general eye irritation, and can also be used to treat another eye disorder if it contains a drug for that disorder. Such formulations provide a comfortable ophthalmic formulation when instilled in the eye and have enhanced efficacy and duration of action over formulations of NSAIDs that are not combined with such agents. Preferably, the effective amount of NSAID present in the formulations of the present invention is sufficient to reduce the discomfort associated with chronic dry eye and/or ocular irritation, but is below that level which would cause an anesthetic effect.

The extraordinary efficacy of these formulations is attributed to, among other things, the synergistic effect of the combination of ingredients in them. The combination of an NSAID and tear substitute, or one or more components thereof, act synergistically to treat signs and symptoms of dry eye, which has never been previously contemplated to be accomplished in one product containing the two separate ingredients. The tear substitute component(s) enhances the integrity of the tear film thereby providing protection of the ocular surface (e.g., by increasing the tear film break-up time and/or the ocular protection index). The NSAID reduces ocular discomfort associated with dry eye. As such, the compositions of the invention are comfortable upon instillation into the eye, and may be used for relief of acute or chronic dry eye disease, and are particularly suitable for both intermittent and long term use. The concentration of the NSAID is at a level that is sufficient to reduce discomfort, without creating an anesthetic effect. The concentration of the NSAID in combination with the tear substitute component(s) also is at a level which would be expected to have reduced adverse effects associated with current FDA approved formulations of NSAIDs marketed for the treatment of acute ocular inflammation and pain, including without limitation, corneal damage, delayed wound healing, and ocular discomfort. As such, the comfortable ophthalmic formulations described herein will treat signs and symptoms of dry eye, increase patient compliance in the use of such formulations for the treatment and/or prevention of signs and symptoms associated with dry eye disease and/or ocular discomfort.

Exemplary NSAIDs suitable for use in the compositions of the invention include, but are not limited to, agents that inhibit the cycloxygenase (COX)-1 and/or -2 enzyme, including but not limited to propionic acids such as naproxen, flurbiprofen, oxaprozin, ibuprofen, ketoprofen, fenoprofen; ketorolac tromethamine (and the other compounds described as being opthalmologically effective in U.S. Pat. No. 4,454,151 to Waterbury, issued Jun. 12, 1984, the pertinent portions of which are incorporated herein by reference); acetic acid derivatives such as sulindac, indomethacin, and etodolac; phenylacetic acids such as diclofenac (and the other compounds described as being opthalmologically effective in U.S. Pat. No. 4,960,799 to Nagy, issued Oct. 2, 1990, the pertinent portions of which are incorporated herein by reference), bromfenac, and suprofen; arylacetic prodrugs such as nepafenac, and amfenac; salicyclic acids, such as aspirin, salsalate, diflunisal, choline magnesium trisalicylate (CMT); para-aminophenol derivatives such as acetaminophen; naphthylalkanones such as nabumetone; enolic acid derivatives such as piroxicam and meloxicam; femanates such as mefenamic acid, meclofenamate and flufenamic acid; pyrroleacetic acids such as tolmetin; and pyrazolones such as phenylbutazone; COX-2 selective inhibitors such as celecoxib, valdecoxib, parecoxib, etoricoxib, and luaricoxib; including all esters and pharmaceutically acceptable salts thereof.

The compositions of the invention comprise a low dose NSAID in an amount effective to relieve acute or chronic corneal discomfort without causing anesthesia or damage to the cornea upon repeated, long term administration. Anesthesia can be measured in the eye using methods known in the art, such as Cochet-Bonnet testing, or other type of esthesiometer. “Low dose NSAID” as used herein refers to a dose lower than current FDA approved ophthalmic NSAID formulations marketed for the treatment of acute ocular inflammation, e.g., inflammation associated with post-ocular surgery. For example, dosages of ophthalmic NSAID formulations currently marketed for the treatment of acute ocular inflammation and pain are as follows: ketorolac tromethamine 0.5% (Acular®), ketorolac tromethamine 0.4% (Acular LS®), diclofenac 0.1% (Voltaren®), bromfenac 0.09% (Xibrom®), nepafenac 0.1% (Nevanac®), flurbiprofen 0.03% (Ocufen®), and suprofen 1%. Preferably, the low dose NSAID in the pharmaceutical compositions of the invention is about 10-80%, more preferably about 30-80%, even more preferably about 40-65% of the dose of ophthalmic NSAID formulations marketed for the treatment of acute ocular inflammation.

The pharmaceutical ophthalmic formulations of the invention typically contain an effective, low dosage amount, e.g., 0.001% to 1% wt/vol, preferably about 0.003% to 0.8% of an active ingredient (e.g., the NSAID), suitable for short and long term use for the treatment of acute or chronic conditions. The amount of active ingredient will vary with the particular formulation and the disease state for which it is intended. For example, effective amounts of ketorolac tromethamine (also referred to herein as ketorolac) range from about 0.04% to about 0.3%, preferably about 0.10% to about 0.3%, more preferably about 0.15% to about 0.30%, even more preferably about 0.15% to about 0.26%; effective amounts of flurbiprofen range from about 0.003% to about 0.024%, preferably about 0.009% to about 0.024%, more preferably about 0.012% to about 0.0195%; effective amounts of nepafenac range from about 0.01% to about 0.08%, preferably about 0.03% to about 0.08%, more preferably about 0.04% to about 0.065%; effective amounts of suprofen range from about 0.3% to about 0.8%, more preferably about 0.4% to about 0.65%; effective amounts of bromfenac range from about 0.009% to about 0.072%, preferably about 0.027% to about 0.072%, more preferably about 0.036% to about 0.059%; effective amounts of diclofenac range from about 0.01% to about 0.08%, preferably about 0.03% to about 0.08%, more preferably about 0.04% to about 0.065%; and effective amounts of indomethacin range from about 0.01% to about 0.1%, preferably about 0.03% to about 0.08%, more preferably about 0.04% to about 0.065%.

A variety of tear substitute components are known in the art and include, but are not limited to: polyols such as, glycerol, glycerin, polyethylene glycol 300, polyethylene glycol 400, polysorbate 80, propylene glycol, and ethylene glycol, polyvinyl alcohol, povidone, and polyvinylpyrrolidone; cellulose derivatives such hydroxypropyl methyl cellulose (also known as hypromellose), carboxymethyl cellulose sodium, hydroxypropyl cellulose, hydroxyethyl cellulose, and methyl cellulose; dextrans such as dextran 70; water soluble proteins such as gelatin; carbomers such as carbomer 934P, carbomer 941, carbomer 940 and carbomer 974P; and gums such as HP-guar, or combinations thereof.

Many tear substitutes containing such components are commercially available, which include, but are not limited to cellulose esters such as Bion Tears®, Celluvisc®, GenTeal®, OccuCoat®, Refresh®, Teargen II®, Tears Naturale®, Tears Naturale®, Tears Naturale Free®, and TheraTears®; and polyvinyl alcohols such as Akwa Tears®, HypoTears®, Moisture Eyes®, Murine Lubricating®, Systane® Lubricant Eye Drops, and Visine Tears®. Tear substitutes may also be comprised of paraffins, such as the commercially available Lacri-Lube® ointments. Other commercially available ointments that are used as tear substitutes include Lubrifresh PM®, Moisture Eyes PM® and Refresh PM®.

In a preferred embodiment, the tear substitute, or one or more components thereof, is an aqueous solution having a viscosity in a range which optimizes efficacy of supporting the tear film while minimizing blurring, lid caking, etc. Preferably, the viscosity of the tear substitute, or one or more components thereof, ranges from 30-150 centipoise (cpi), preferably 30-130 cpi, more preferably 50-120 cpi, even more preferably 70-120 cpi. In some embodiments, the viscosity of the tear substitute, or one or more components thereof, ranges from about 50-90 cpi.

In some embodiments, the tear substitute, or one or more components thereof is buffered to a pH 5.0 to 9.0, preferably pH 5.5 to 8.5, more preferably pH 6 to 8, with a suitable salt (e.g., phosphate salts). In some embodiments, the tear substitute further comprises one or more ingredients, including without limitation, glycerol, propyleneglycerol, glycine, sodium borate, magnesium chloride, and zinc chloride.

In one preferred embodiment of the invention, the tear substitute comprises hydroxypropylmethyl cellulose. For example, without limitation, a tear substitute which comprises hydroxypropyl methyl cellulose is GenTeal® lubricating eye drops. GenTeal® (CibaVision-Novartis) is a sterile lubricant eye drop containing hydroxypropylmethyl cellulose 3 mg/g and preserved with sodium perborate. Other examples of an HPMC-based tear are provided. Preparation and use of one such tear is described in U.S. Pat. No. 6,806,364, which is expressly incorporated by reference herein in its entirety. This tear contains 0.2 to 2.5 (e.g., 0.5 to 0.8) percent by weight of hydroxypropylmethyl cellulose, 0.045 to 0.065 (e.g., 0.05 to 0.06) percent by weight a calcium salt, and 0.14 to 1.4 (e.g., 0.3 to 1.2) percent by weight a phosphate salt. This tear has a viscosity of 20 to 150 (e.g., 50 to 90) centipoise and is buffered to a pH 5.5 to 8.5 (e.g., 6 to 8) with a phosphate salt or other suitable salts. It may further contain one or more of the following ingredients: 0.5 to 1.0 percent by weight glycerol, 0.5 to 1.0 percent by weight propyleneglycerol, 005 to 0.05 percent by weight glycine, 0.006 to 0.08 percent by weight sodium borate, 0.025 to 0.10 percent by weight magnesium chloride, and 0.001 to 0.01 percent by weight zinc chloride.

In another preferred embodiment, the tear substitute comprises carboxymethyl cellulose sodium. For example, without limitation, the tear substitute which comprises carboxymethyl cellulose sodium is Refresh® Tears. Refresh® Tears is a lubricating formulation similar to normal tears, containing a, mild non-sensitizing preservative, stabilised oxychloro complex (Purite™), that ultimately changes into components of natural tears when used.

In certain embodiments, the one or more tear substitute components acts as the pharmaceutical carrier(s).

In certain embodiments, the pharmaceutical compositions of the invention may comprise combinations of at least two NSAIDs and one or more tear substitute components. In other embodiments, the topical formulations of the invention may comprise one or more anti-allergenic agents and a combination of one or more tear substitute components.

The pharmaceutical compositions of the invention described above may additionally comprise other active ingredients, including, but not limited to, vasoconstrictors, anti-allergenic agents, anti-infectives, steroids, anesthetics, anti-inflammatories, analgesics, dry eye agents (e.g. secretagogues, mucomimetics, polymers, lipids, antioxidants), etc., or be administered in conjunction (simultaneously or sequentially) with pharmaceutical compositions comprising other active ingredients, including, but not limited to, vasoconstrictors, anti-allergenic agents, anti-infectives, steroids, anesthetics, anti-inflammatories, analgesics, dry eye agents (e.g. secretagogues, mucomimetics, polymers, lipids, antioxidants), etc.

For example, the NSAID/tear substitute compositions of the invention may be used in combination with another pharmaceutical composition, such as a prescription drug like Restasis™ (cyclosporine ophthalmic emulsion, 0.05%). It may be used simultaneously with another pharmaceutical composition, or in sequence. For example, the NSAID/tear substitute compositions of the invention may be administered to a subject in the ramp up period before another administered pharmaceutical begins to be effective in the subject. In certain embodiments, the NSAID/tear substitute compositions of the invention may be used in a manner such that they serve as a replacement for a prescription drug like Restasis™.

The NSAIDs and other active ingredients of the pharmaceutical compositions may be in the form of a pharmaceutically acceptable salt.

Preferably, the pharmaceutical compositions according to the present invention will be formulated as solutions, suspensions, ointments, gels, sustained release formulation, and other dosage forms for topical administration or for sustained release delivery. Aqueous solutions are generally preferred, based on ease of formulation, as well as a patient's ability to easily administer such compositions by means of instilling one to two drops of the solutions in the affected eyes. However, the compositions may also be suspensions, viscous or semi-viscous gels, or other types of solid or semi-solid compositions, or those appropriate for sustained release.

Any of a variety of carriers may be used in the formulations of the present invention including water, mixtures of water and water-miscible solvents, such as C₁-to C₇-alkanols, vegetable oils or mineral oils comprising from 0.5 to 5% non-toxic water-soluble polymers, natural products, such as gelatin, alginates, pectins, tragacanth, karaya gum, xanthan gum, carrageenin, agar and acacia, starch derivatives, such as starch acetate and hydroxypropyl starch, and also other synthetic products, such as polyvinyl alcohol, polyvinylpyrrolidone, polyvinyl methyl ether, polyethylene oxide, preferably cross-linked polyacrylic acid, such as neutral Carbopol, or mixtures of those polymers. The concentration of the carrier is, typically, from 1 to 100000 times the concentration of the active ingredient.

Additional ingredients that may be included in the formulation include tonicity enhancers, preservatives, solubilizers, stabilizers, non-toxic excipients, demulcents, sequestering agents, pH adjusting agents, co-solvents and viscosity building agents.

For the adjustment of the pH, preferably to a physiological pH, buffers may especially be useful. The pH of the present solutions should be maintained within the range of 4.0 to 8.0, more preferably about 4.0 to 6.0, more preferably about 6.5 to 7.8. Suitable buffers may be added, such as boric acid, sodium borate, potassium citrate, citric acid, sodium bicarbonate, TRIS, and various mixed phosphate buffers (including combinations of Na₂HPO₄, NaH₂PO₄ and KH₂PO₄) and mixtures thereof. Generally, buffers will be used in amounts ranging from about 0.05 to 2.5 percent by weight, and preferably, from 0.1 to 1.5 percent.

Tonicity is adjusted if needed typically by tonicity enhancing agents. Such agents may, for example be of ionic and/or non-ionic type. Examples of ionic tonicity enhancers are alkali metal or earth metal halides, such as, for example, CaCl₂, KBr, KCl, LiCl, NaI, NaBr or NaCl, Na₂SO₄ or boric acid. Non-ionic tonicity enhancing agents are, for example, urea, glycerol, sorbitol, mannitol, propylene glycol, or dextrose. The aqueous solutions of the present invention are typically adjusted with tonicity agents to approximate the osmotic pressure of normal lachrymal fluids which is equivalent to a 0.9% solution of sodium chloride or a 2.5% solution of glycerol. An osmolality of about 225 to 400 mOsm/kg is preferred, more preferably 280 to 320 mOsm.

In certain embodiments, the formulations of the invention additionally comprise a preservative. A preservative may typically be selected from a quaternary ammonium 30 compound such as benzalkonium chloride, benzoxonium chloride or the like. Benzalkonium chloride is better described as: N-benzyl-N—(C₈-C₁₈ alkyl)-N,Ndimethylammonium chloride. Examples of preservatives different from quaternary ammonium salts are alkyl-mercury salts of thiosalicylic acid, such as, for example, thiomersal, phenylmercuric nitrate, phenylmercuric acetate or phenylmercuric borate, sodium perborate, sodium chlorite, parabens, such as, for example, methylparaben or propylparaben, alcohols, such as, for example, chlorobutanol, benzyl alcohol or phenyl ethanol, guanidine derivatives, such as, for example, chlorohexidine or polyhexamethylene biguanide, sodium perborate, Germal®II or sorbic acid. Preferred preservatives are quaternary ammonium compounds, in particular benzalkonium chloride or its derivative such as Polyquad (see U.S. Pat. No. 4,407,791), alkyl-mercury salts and parabens. Where appropriate, a sufficient amount of preservative is added to the ophthalmic composition to ensure protection against secondary contaminations during use caused by bacteria and fungi.

In other embodiments, the formulations of this invention do not include a preservative. Such formulations would be useful for patients with dry eye, patients who wear contact lenses, or those who use several topical ophthalmic drops and/or those with an already compromised ocular surface (e.g. dry eye) wherein limiting exposure to a preservative may be more desirable.

The formulation of the invention may additionally require the presence of a solubilizer, in particular if the active or the inactive ingredients tends to form a suspension or an emulsion. A solubilizer suitable for an above concerned composition is for example selected from the group consisting of tyloxapol, fatty acid glycerol polyethylene glycol esters, fatty acid polyethylene glycol esters, polyethylene glycols, glycerol ethers, a cyclodextrin (for example alpha-, beta- or gamma-cyclodextrin, e.g. alkylated, hydroxyalkylated, carboxyalkylated or alkyloxycarbonyl-alkylated derivatives, or mono- or diglycosyl-alpha-, beta- or gamma-cyclodextrin, mono- or dimaltosyl-alpha-, beta- or gamma-cyclodextrin or panosyl-cyclodextrin), polysorbate 20, polysorbate 80 or mixtures of those compounds. A specific example of an especially preferred solubilizer is a reaction product of castor oil and ethylene oxide, for example the commercial products Cremophor EL® or Cremophor RH40®. Reaction products of castor oil and ethylene oxide have proved to be particularly good solubilizers that are tolerated extremely well by the eye. Another preferred solubilizer is selected from tyloxapol and from a cyclodextrin. The concentration used depends especially on the concentration of the active ingredient. The amount added is typically sufficient to solubilize the active ingredient. For example, the concentration of the solubilizer is from 0.1 to 5000 times the concentration of the active ingredient.

The formulations may comprise further non-toxic excipients, such as, for example, emulsifiers, wetting agents or fillers, such as, for example, the polyethylene glycols designated 200,300,400 and 600, or Carbowax designated 1000, 1500,4000,6000 and 10000. The amount and type of excipient added is in accordance with the particular requirements and is generally in the range of from approximately 0.0001 to approximately 90% by weight.

Other compounds may also be added to the formulations of the present invention to increase the viscosity of the carrier. Examples of viscosity enhancing agents include, but are not limited to: polysaccharides, such as hyaluronic acid and its salts, chondroitin sulfate and its salts, dextrans, various polymers of the cellulose family; vinyl polymers; and acrylic acid polymers.

2. Packaging

The formulations of the present invention may be packaged as either a single dose product or a multi-dose product. The single dose product is sterile prior to opening of the package and all of the composition in the package is intended to be consumed in one or several applications to one or both eyes of a patient. The use of an antimicrobial preservative to maintain the sterility of the composition after the package is opened is generally unnecessary. The formulations, if an ointment formulation, may be packaged as appropriate for an ointment, as is known to one of skill in the art.

Multi-dose products are also sterile prior to opening of the package. However, because the container for the composition may be opened many times before all of the composition in the container is consumed, the multi-dose products must have sufficient antimicrobial activity to ensure that the compositions will not become contaminated by microbes as a result of the repeated opening and handling of the container. The level of antimicrobial activity required for this purpose is well known to those skilled in the art, and is specified in official publications, such as the United States Pharmacopoeia (“USP”) and other publications by the Food and Drug Administration, and corresponding publications in other countries. Detailed descriptions of the specifications for preservation of ophthalmic pharmaceutical products against microbial contamination and the procedures for evaluating the preservative efficacy of specific formulations are provided in those publications. In the United States, preservative efficacy standards are generally referred to as the “USP PET” requirements. (The acronym “PET” stands for “preservative efficacy testing.”)

The use of a single dose packaging arrangement eliminates the need for an anti-microbial preservative in the compositions, which is a significant advantage from a medical perspective, because conventional antimicrobial agents utilized to preserve ophthalmic compositions (e.g., benzalkonium chloride) may cause ocular irritation, particularly in patients suffering from dry eye conditions or pre-existing ocular irritation, or patients using multiple preserved products. However, the single dose packaging arrangements currently available, such as small volume plastic vials prepared by means of a process known as “form, fill and seal”, have several disadvantages for manufacturers and consumers. The principal disadvantages of the single dose packaging systems are the much larger quantities of packaging materials required, which is both wasteful and costly, and the inconvenience for the consumer. Also, there is a risk that consumers will not discard the single dose containers following application of one or two drops to the eyes, as they are instructed to do, but instead will save the opened container and any composition remaining therein for later use. This improper use of single dose products creates a risk of microbial contamination of the single dose product and an associated risk of ocular infection if a contaminated composition is applied to the eyes.

While the formulations of this invention are preferably formulated as “ready for use” aqueous solutions, alternative formulations are contemplated within the scope of this invention. Thus, for example, the active ingredients, surfactants, salts, chelating agents, or other components of the ophthalmic solution, or mixtures thereof, can be lyophilized or otherwise provided as a dried powder or tablet ready for dissolution (e.g., in deionized, or distilled) water. Because of the self-preserving nature of the solution, sterile water is not required.

3. Methods of Use

The invention features methods of treating and/or preventing the signs and symptoms associated with dry eye and/or eye irritation in a subject comprising use of the novel formulations described above. For example, a method of treating and/or preventing dry eye and/or eye irritation may comprise administering to the eye surface of the subject in need thereof a formulation comprising an effective amount of at least one NSAID and a tear substitute, or one or more components thereof, in a pharmaceutically acceptable carrier.

Provided also are methods of increasing the tear film break-up time (TFBUT) of a subject's tear film, comprising administering to the eye surface of the subject in need thereof a formulation comprising an effective amount of at least one NSAID and a tear substitute, or one or more components thereof, in a pharmaceutically acceptable carrier.

Provided also are methods of increasing the ocular protection index (OPI) of a subject's eye, comprising administering to the eye surface of the subject in need thereof a formulation comprising an effective amount of at least one NSAID and a tear substitute, or one or more components thereof, in a pharmaceutically acceptable carrier.

Provided also are methods for improving, treating, relieving, inhibiting, preventing, or otherwise decreasing ocular discomfort in a subject comprising administering to the eye surface of the subject in need thereof a formulation comprising an effective amount of at least one NSAID and a tear substitute, or one or more components thereof, in a pharmaceutically acceptable carrier.

Additionally provided are methods for decreasing the adverse effects associated with the administration of an NSAID to the eye, comprising administering to the eye surface of the subject in thereof a formulation comprising an effective amount of at least one NSAID and a tear substitute, or one or more components thereof. Examples of an adverse effect associated with the administration of an NSAID to the eye include but are not limited to corneal damage, delayed wound healing, and ocular discomfort.

The effective amount of NSAIDs in the formulation will depend on absorption, inactivation, and excretion rates of the drug as well as the delivery rate of the compound from the formulation, and will be suitable for short or long term use for the treatment of acute or chronic conditions, respectively. It is to be noted that dosage values may also vary with the severity of the condition to be alleviated. It is to be further understood that for any particular subject, specific dosage regimens should be adjusted over time according to the individual need and the professional judgment of the person administering or supervising the administration of the compositions. Typically, dosing will be determined using techniques known to one skilled in the art.

The dosage of any compound of the present invention will vary depending on the symptoms, age and other physical characteristics of the patient, the nature and severity of the disorder to be treated or prevented, the degree of comfort desired, the route of administration, and the form of the supplement. Any of the subject formulations may be administered in a single dose or in divided doses. Dosages for the formulations of the present invention may be readily determined by techniques known to those of skill in the art or as taught herein.

An effective dose or amount, and any possible effects on the timing of administration of the formulation, may need to be identified for any particular formulation of the present invention. This may be accomplished by routine experiment as described herein. The effectiveness of any formulation and method of treatment or prevention may be assessed by administering the formulation and assessing the effect of the administration by measuring one or more indices associated with the efficacy of the NSAID composition and with the degree of comfort to the patient, as described herein, and comparing the post-treatment values of these indices to the values of the same indices prior to treatment or by comparing the post-treatment values of these indices to the values of the same indices using a different formulation.

The precise time of administration and amount of any particular formulation that will yield the most effective treatment in a given patient will depend upon the activity, pharmacokinetics, and bioavailability of a particular compound, physiological condition of the patient (including age, sex, disease type and stage, general physical condition, responsiveness to a given dosage and type of medication), route of administration, and the like. The guidelines presented herein may be used to optimize the treatment, e.g., determine the optimum time and/or amount of administration, which will require no more than routine experimentation consisting of monitoring the subject and adjusting the dosage and/or timing.

The combined use of several NSAIDs formulated into the compositions of the present invention may reduce the required dosage for any individual component because the onset and duration of effect of the different components may be complimentary. In such combined therapy, the different NSAIDs may be delivered together or separately, and simultaneously or at different times within the day.

Efficacy of the formulations and compositions of the invention in treating and preventing the signs and symptoms associated with dry eye disease and/or ocular irritation may be assessed by measuring changes in tear film break-up time, changes in ocular protection index, and level of ocular comfort. An increase in tear film break-up time and/or ocular protection index in a subject, following administration of the formulations and compositions of the invention as compared to TFBUT and or OPI prior to administration, indicates that the formulation is effective in treating and preventing signs and symptoms associated with dry eye disease and/or ocular irritation. TFBUT may be measured using various methods, including but not limited to illumination of the eye following instillation of sodium fluorescein in the eye, or equivalents thereof. An increase in ocular comfort or decrease in ocular discomfort in a subject following administration of the formulations and compositions of the invention as compared to ocular comfort level prior to administration, indicates that the formulation is effective in treating and preventing signs and symptoms associated with dry eye disease and/or ocular irritation. Ocular comfort level may be assessed by various methods, including but not limited to subjective scales (for example but not limited to, standardized subjective scales that determine ocular discomfort as mild, moderate, severe, or 0, 1, 2, 3, 4, etc., or other appropriate scale), reflexive response (e.g., flinch-reflex), and physiological response, including but not limited to changes in heart rate, blood pressure, and perspiration levels.

4. Kits

In still another embodiment, this invention provides kits for the packaging and/or storage and/or use of the formulations described herein, as well as kits for the practice of the methods described herein. Thus, for example, kits may comprise one or more containers containing one or more ophthalmic solutions, ointments, gels, sustained release formulations or devices, suspensions or formulations, tablets, or capsules of this invention. The kits can be designed to facilitate one or more aspects of shipping, use, and storage.

The kits may optionally include instructional materials containing directions (i.e., protocols) disclosing means of use of the formulations provided therein. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g. CD ROM), and the like. Such media may include addresses to internet sites that provide such instructional materials.

All publications and patents mentioned herein are hereby incorporated by reference in their entirety as if each individual publication or patent was specifically and individually indicated to be incorporated by reference. In case of conflict, the present application, including any definitions herein, will control.

### EXAMPLES

The invention now being generally described, it will be more readily understood by reference to the following examples which are included merely for purposes of illustration of certain aspects and embodiments of the present invention, and are not intended to limit the invention in any way.

### Example 1

### Formulation of Acular (Ketorolac Tromethamine) with a Carboxymethyl Cellulose (CMC)-Based Artificial Tear

The following study compared the efficacy of ketorolac tromethamine 0.5% ophthalmic solution

(Acular®), Acular® combined with a CMC-based artificial tear (Refresh®) (1:1 dilution, final concentration ketorolac tromethamine 0.25%), and Refresh® alone, in reducing ocular discomfort.

A specially developed chamber called the controlled adverse environment (CAE) was used as a model for evaluating ocular discomfort caused by irritation. The CAE is a chamber in which humidity is controlled at a low level, and temperature, wind flow, lighting and visual tasking are all controlled. Patients who enter the CAE will develop ocular discomfort over time. This model allows for the precise evaluation of agents which can act to treat dry eye and/or ocular irritation.

Baseline ocular exams were performed by an ophthalmologist on eighteen subjects. Subjects then entered the CAE and remained for 60 minutes. Every 5 minutes the ocular discomfort of each eye was assessed by the subject on a standardized 0-4 ocular discomfort scale, and was recorded by study staff. When an eye manifested a score of at least 3 at two consecutive assessments, 1-2 drops of either ketorolac tromethamine 0.5% ophthalmic solution, combined ketorolac tromethamine 0.25%/Refresh® formulation, or placebo (Refresh® artificial tear alone), was instilled into the eye. Subjects recorded comfort of the drop immediately following instillation of the drop on a 0-9 comfort scale (0=extremely comfortable and 9=extremely uncomfortable) and remained in the CAE 90 more minutes, with ocular discomfort assessments.

Each eye was dosed and assessed separately when it reached a score of at least 3 at two consecutive measurements during the initial CAE exposure.

An exit ocular exam was performed following the 90 minute follow-up CAE exposure by an ophthalmologist.

Ketorolac tromethamine 0.5% ophthalmic solution (N=8 eyes) showed a reduction in ocular discomfort scores compared with placebo (N=7 eyes) following dosing when subjects were exposed to the CAE. The reduction was evident starting at 15 minutes of exposure in the CAE postdosing. The ketorolac 0.25%/Refresh® combined formulation (N=5) also showed a reduction in ocular discomfort scores compared with placebo (N=5) following dosing when subjects were exposed to the CAE. The reduction with the combined ketorolac 0.25%/Refresh® formulation was evident at 40 minutes post-instillation of treatment. While the effect of the combined ketorolac 0.25%/Refresh® formulation was less than that of ketorolac 0.5%, there was still evidence that the combined ketorolac 0.25%/Refresh® formulation reduced discomfort.

The comfort of the drop immediately following instillation in the eye was superior in the placebo and the combined ketorolac 0.25%/Refresh® formulation treated eyes than the ketorolac 0.5% ophthalmic solution treated eyes. There was no difference between the comfort of the combined ketorolac 0.25%/Refresh® formulation and placebo drops. Thus, a drop consisting of a concentration less than currently available Acular® (0.5% ketorolac ophthalmic solution) was more comfortable when placed in the eye but still acted to treat ocular discomfort due to irritation. It can be expected that further dose range testing can identify a concentration higher than 0.25% but less than 0.5% which is more comfortable than 0.5% but is more efficacious than 0.25%. Other concentrations with these characteristics are also intended to be encompassed in this invention.

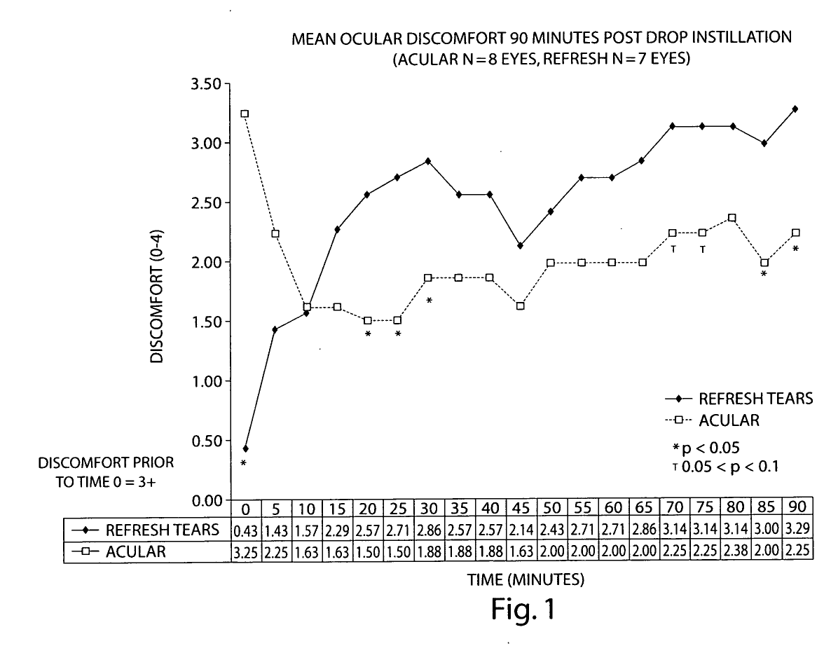

The data (FIG. 1) shows that a concentration of a topical NSAID can be identified which is able to reduce ocular discomfort.

### Example 2

### Formulation of Acular® (Ketorolac Tromethamine 0.5% Ophthalmic Solution) with a Hydroxypropylmethyl Cellulose (HPMC)-Based Artificial Tear

The following study compares the efficacy of an HPMC-based artificial tear with a combined formulation of Acular® and an HPMC-based artificial tear (1:1 dilution, final concentration ketorolac 0.25%), in reducing ocular discomfort.

Baseline ocular exams were performed by an ophthalmologist on eight subjects. Subjects then entered the CAE (described in Example 1) and remained for up to 90 minutes. Every 5 minutes the ocular discomfort of each eye was assessed by the subject on a standardized 0-4 ocular discomfort scale, and was recorded by study staff. When an eye manifested a score of at least 3 at two consecutive assessments, 1-2 drops of the HPMC-based tear was instilled in one eye and 1-2 drops of the combined Acular®/HPMC-based tear formulation in the contralateral eye. Subjects recorded comfort of the drop immediately following instillation of the drop on a 0-9 comfort scale (0=extremely comfortable and 9=extremely uncomfortable) and remained in the CAE 60 more minutes, with ocular discomfort assessments every 5 minutes.

Each eye was dosed and assessed separately when it reached a score of at least 3 at two consecutive measurements during the initial CAE exposure.

An exit ocular exam was performed following the 60 minute follow-up CAE exposure by an ophthalmologist.

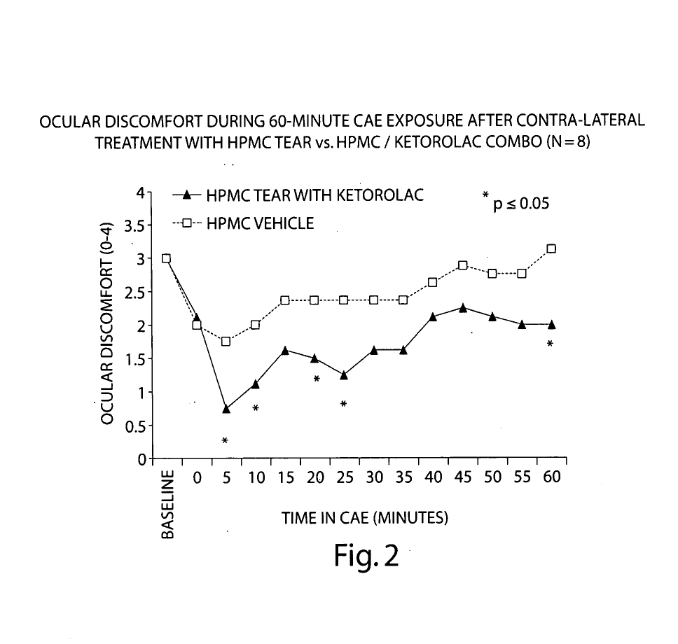

FIG. 2 depicts the results of this study. The combined Acular®/HPMC-based tear formulation (final concentration ketorolac 0.25%) significantly improved ocular discomfort during CAE challenge. The HPMC-based tear reduced the ocular stinging typically associated with ketorolac upon instillation.

### Example 3

### Tear Film Break-Up Time (TFBUT)

The “tear film break-up time” or “TFBUT” test, an index of the severity of dry eye syndrome, can be used to measure the efficacy of a solution in maintaining the tear film. It is correlated with the degree of ocular discomfort a subject may feel. In a study involving hundreds of subjects, over 70% reported ocular discomfort within 1 second of tear film break-up. On average, the tear film in a normal eye breaks up in 7.1 seconds. In contrast, the tear film in a “dry eye” breaks up in an average of 3.2 seconds. Thus, agents having the ability to increase the TFBUT could be used in treating and preventing dry eye.

For example, the TFBUT may be assessed as follows. A patient's eye is first instilled with 2% sodium fluorescein. After the fluorescein instillation, the patient places his or her head in a slit lamp, and the investigator views the eye under cobalt blue illumination. The patient is instructed to blink three times and hold the eyes open at normal aperture after the third blink.

A stop watch is started when the eye is opened following the third blink, and is stopped when the investigator identifies a region of tear film break-up that has started to expand. The region of tear film break-up is identifiable by black voids in the otherwise confluent appearing tear film. The eye is video taped during the test.

The efficacy of the ophthalmic solutions described in Examples 1 and 2 on the TFBUT in dry-eye patients may be tested as follows. First, a TFBUT baseline for each patient is established. One or two drops of the ophthalmic formulation is then applied into one eye of each patient and the TFBUT is measured at 5, 10, 15, 30, 45, and 60 minutes after the application.

The TFBUT may be used to derive an ocular protection index (OPI) (Nally L, Ousler G W, Abelson M B. Ocular discomfort and tear film break-up time in dry eye 25 patients: a correlation. IOVS 2000 41; 4 (ARVO Abstract): 1436.), which is obtained by dividing the TFBUT by the time in seconds between blinks (the inter-blink interval, or “IBI”). An OPI of 1 or more than 1 (that is, the TFBUT is greater than or equal to the IBI) indicates a tear-protected ocular surface, with minimized signs or symptoms of dry eye. An OPI of less than 1 (that is, the TFBUT is less than the IBI) indicates an unprotected ocular surface, with exacerbated signs or symptoms of dry eye.

### Example 4

### Formulation of Various NSAIDs with an HPMC-Based Artificial Tear

The following study compares the efficacy of an HPMC-based artificial tear with various 1:1 tear:NSAID combined formulations. The following commercially available ophthalmic NSAIDS were diluted 1:1 with an HPMC-based artificial tear: ketorolac tromethamine 0.4% (Acular-LS®), yielding an effective concentration of ketorolac tromethamine 0.2%; diclofenac 0.1% (Voltaren®) yielding an effective concentration of diclofenac 0.05%; bromfenac 0.09% (Xibrom®) yielding an effective concentration of 0.045%; and nepafenac 0.1% (Nevanac®), yielding an effective concentration of nepafenac 0.05%.

The HPMC-based tear used in this study was comprised of 0.8% sodium chloride, 0.7% HPMC K4M, and had a pH of 7.4±0.1, and a viscosity of 50 centipoise (cpi).

The CAE chamber described in Example 1 was used as a model for evaluating ocular discomfort caused by irritation. Subjects entered the CAE and remained for up to 30 minutes. Every 5 minutes the ocular discomfort of each eye was assessed by the subject on a standardized 0-4 ocular discomfort scale (0=no discomfort, 4=worst discomfort), and was recorded by study staff. When an eye manifested a score of greater than or equal to 3 at two consecutive assessments, 1-2 drops of the HPMC-based tear was instilled in one eye, and 1-2 drops of the combined NSAID/HPMC-based tear formulation in the contralateral eye, in a randomized fashion. Subjects recorded comfort of the drop immediately following instillation of the drop on a 0-9 comfort scale (0=extremely comfortable and 9=extremely uncomfortable) and remained in the CAE 60 more minutes, with ocular discomfort assessments every 5 minutes.

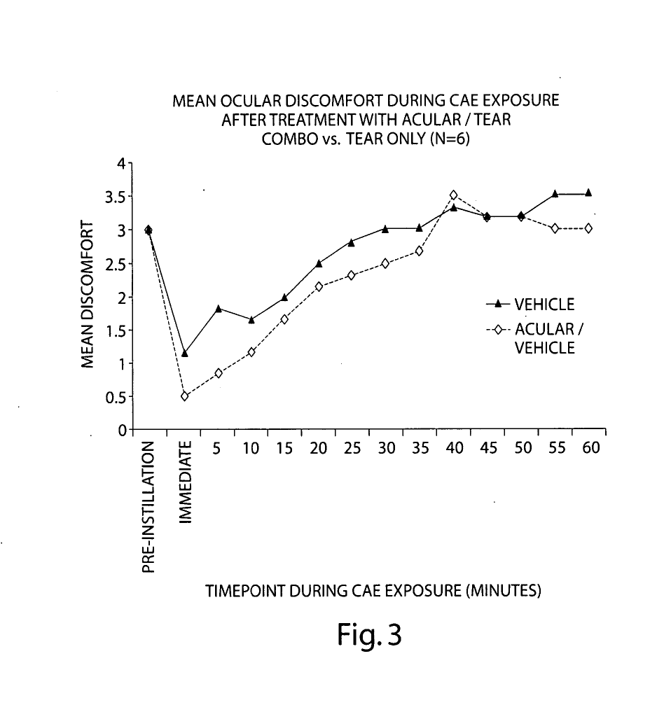

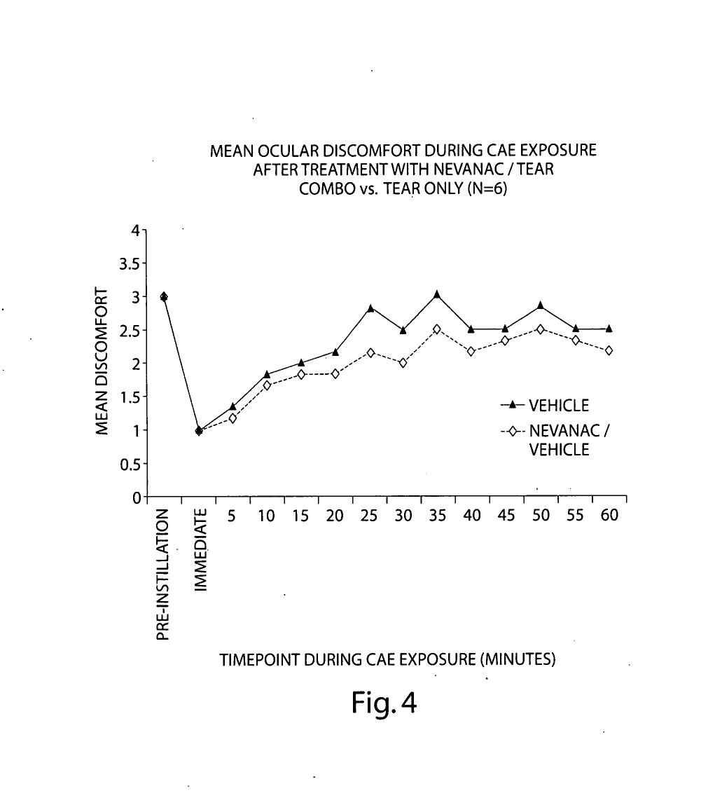

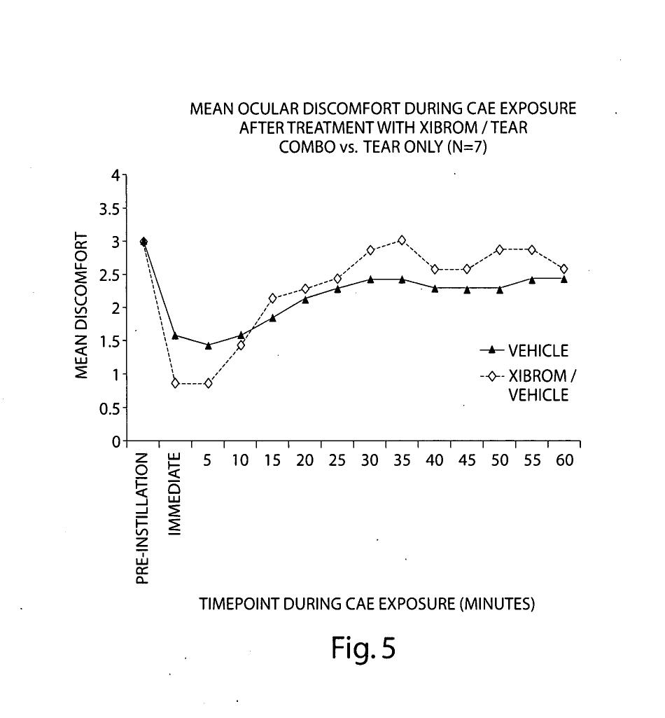

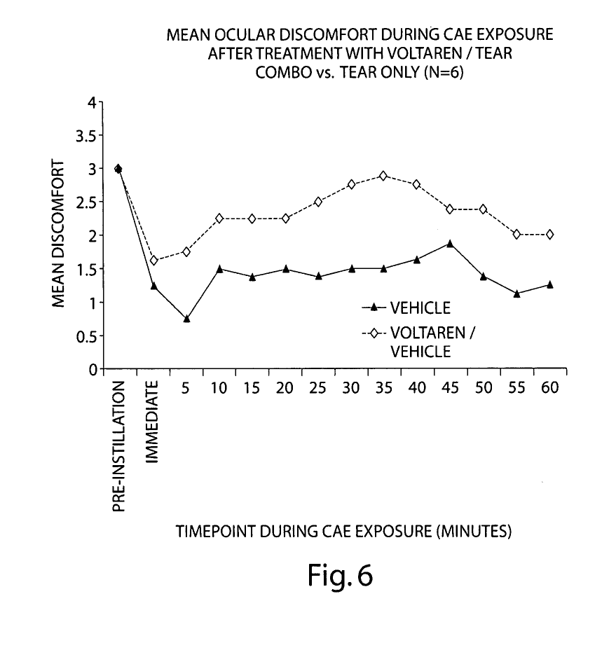

FIGS. 3-6 depict the results of this study. It was observed that 3 of the 4 combined NSAID/HPMC-based tear formulations reduced ocular discomfort during the CAE challenge (see FIGS. 3-5). The combined Acular LS®/HPMC-based tear formulation (final concentration ketorolac 0.2%) yielded the maximal reduction in ocular discomfort compared to the other NSAIDs tested at the given concentrations. While the combined Voltaren®/HPMC-based tear formulation, at the concentration tested, was not more effective than the HPMC-based tear formulation alone (see FIG. 6), the formulation was still effective in reducing ocular discomfort. Additional studies will be designed to test a concentration range of Voltaren® combined with a variety of artificial tears (including but not limited to CMC and HMPC-based artificial tears), ranging in viscosity, to determine the appropriate formulation whereby Voltaren combined with a tear substitute is more efficacious in reducing ocular discomfort in subjects exposed to the CAE challenge.

### Example 5

### Formulation of 0.25%, 0.125%, and 0.06% Ketorolac with an HPMC-Based Artificial Tear

The following study compares the efficacy of various concentrations of ketorolac tromethamine in formulation with an HPMC tear component. The HPMC-based tear used in this study was comprised of 0.8% sodium chloride, 0.72% HPMC K4M, and had a pH of 7.4±0.1, and a viscosity of 70 centipoise (cpi).

Ketorolac tromethamine was formulated at a final concentration of 0.25%, 0.125%, and 0.06% in the HPMC-based artificial tear solution as follows:

| | 0.25% Ketorolac | 0.125% Ketorolac | 0.06% Keterolac |
|---|---|---|---|
| | tromethamine | tromethamine | tromethamine |
| HPMC K4M | 0.72% | 0.72% | 0.72% |
| Sodium | 0.8% | 0.8% | 0.8% |
| Chloride | | | |
| NaOH/HCl | pH 7.4 ± 0.1 | pH 7.4 ± 0.1 | pH 7.4 ± 0.1 |

Note that in the formulations described in the above table, all percentages are percent weight per volume.

The CAE chamber described in Example 1 was used as a model for evaluating ocular discomfort caused by irritation. Subjects entered the CAE and remained for up to 30 minutes. Every 5 minutes the ocular discomfort of each eye was assessed by the subject on a standardized 0-4 ocular discomfort scale (0=no discomfort, 4=worst discomfort), and was recorded by study staff. When an eye manifested a score of greater than or equal to 3 at two consecutive assessments, 1-2 drops of HPMC-based vehicle was instilled in one eye, and 1-2 drops of the ketorolac/HPMC formulation in the contralateral eye, in a randomized fashion. Subjects recorded comfort of the drop immediately following instillation of the drop on a 0-9 comfort scale (0=extremely comfortable and 9=extremely uncomfortable) and remained in the CAE 60 more minutes, with ocular discomfort assessments every 5 minutes.

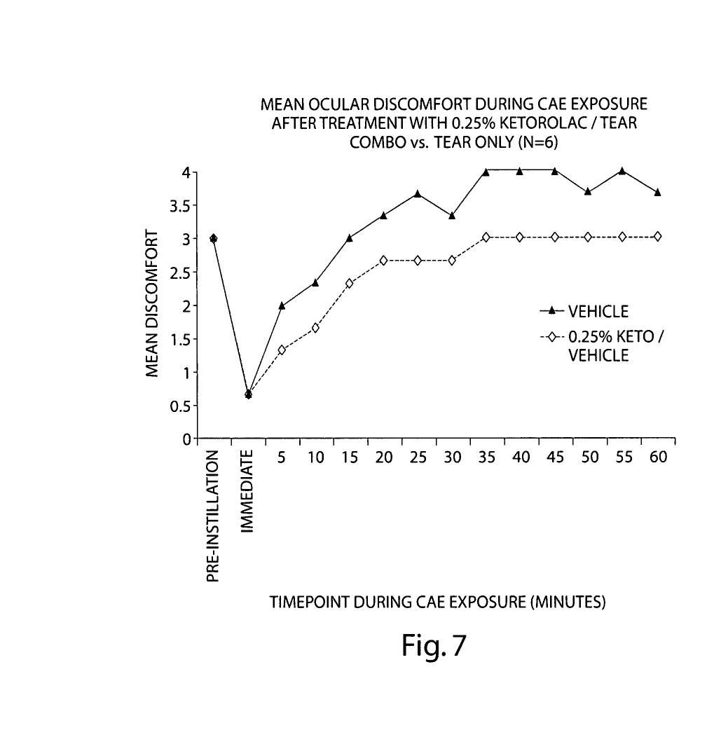

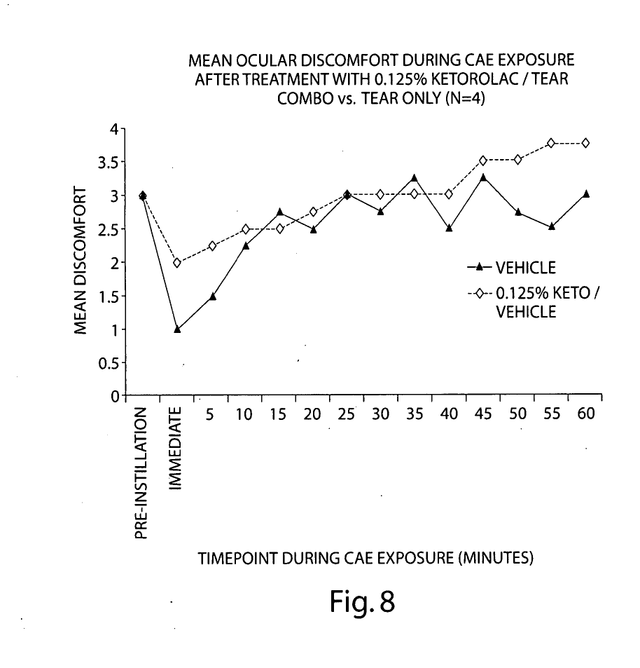

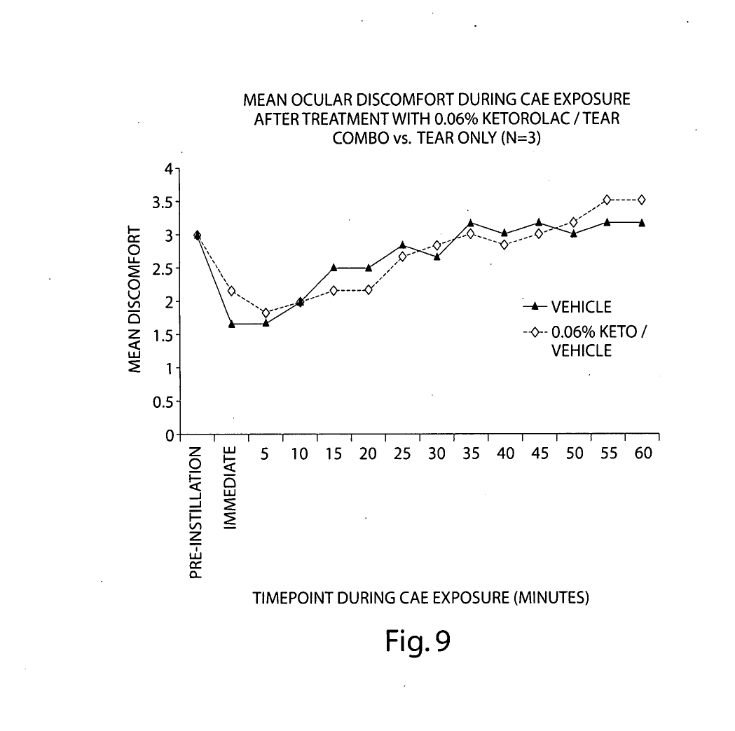

FIGS. 7-9 depicts the results of this study. It was observed that the combined ketorolac 0.25%/HPMC-based tear formulation was efficacious in reducing ocular discomfort while the combined ketorolac 0.125%/tear and ketorolac 0.06%/tear formulations were not. While not intending to be bound by any theory, these results suggest a loss of pharmacologic activity at a point between ketorolac 0.125% and ketorolac 0.25%.

### Example 6

### TFBUT Testing Using Various NSAID/Artificial Tear Formulations

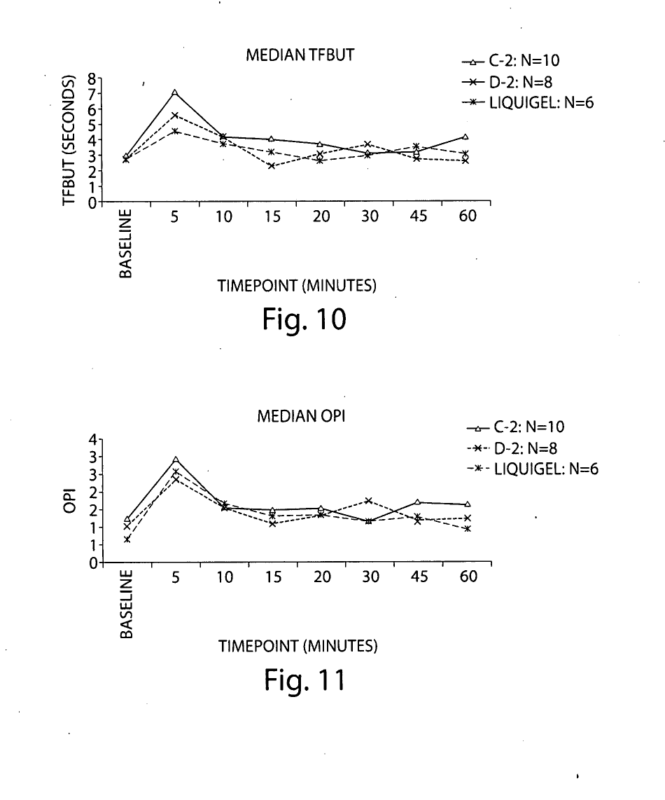

The following study evaluates the efficacy of 3 formulations of artificial tear components (approximately 50-80 cpi) with ketorolac on increasing TFBUT and the Ocular Protection Index (OPI). Two of the tear components were HPMC based (depicted as formulation C-2 and D-2 in the table below, and in FIGS. 10 and 11), and one was CMC based (depicted as Liquigel in the table below and in FIGS. 10 and 11). The formulations used in this study are given in the table below.

| | C-2 | D-2 | Liquigelⁱ |
|---|---|---|---|
| Sodium chloride | | 0.8% | X |
| Sodium phosphate, | 0.34% | | |
| dibasic•7H2O | | | |
| Potassium phosphate, | 0.17% | | |
| monobasic | | | |
| Glycerin | 1.8% | | |
| Magnesium | | | Xⁱⁱ |
| chloride•6H2O | | | |
| HPMC K4M | 0.7% | 0.7% | |
| Ketorolac | 0.25% | 0.25% | 0.25% (1:1 dilution |
| tromethamine | | | with Acular ®- |
| | | | ketorolac 0.5%) |
| Glycine | 0.0075% | | |
| NaOH/HCl | pH 7.4 ± 0.1 | pH 7.4 ± 0.1 | |
| Carboxymethyl- | | | 1.0 |
| cellulose sodium | | | |
| Boric acid | | | X |
| Calcium chloride | | | X |
| Potassium chloride | | | X |
| Sodium borate | | | X |
| PURITE ® (stabilized | | | X |
| oxychloro complex) | | | |
| Purified Water | q.s. 100 | q.s. 100 | X |

ⁱPDR for Ophthalmic Medicines, 35^{th} Edition, 2007
“X” denotes inactive ingredient of unknown percentage
ⁱⁱdoes not clarify 6H2O

1-2 drops of the formulation was instilled in each eye and TFBUT was assessed, as well as blink rate to calculate OPI, as described in Example 3 above. TFBUT was assessed at baseline, and 5, 10, 15, 20, 30, 45, 60 minutes post-dosing. A comparison of each formulation on TFBUT and OPI are shown in FIGS. 10 and 11, respectively.

### Example 7

### Formulation of an NSAID with a Different Artificial Tear

The following study compares the efficacy of an NSAID combined with an artificial tear solution containing the demulcents polyethylene glycol 400 and propylene glycol with HP-Guar (Systane®), in reducing ocular discomfort, with the artificial tear solution alone. The NSAID used in this study was Nevanac®. Systane® was formulated in a 1:1 ratio with the NSAID Nevanac® (nepafenac 0.1%) to yield an effective concentration of 0.5% nepafenac.

The CAE chamber described in Example 1 was used as a model for evaluating ocular discomfort caused by irritation. Subjects entered the CAE and remained for up to 30 minutes. Every 5 minutes the ocular discomfort of each eye was assessed by the subject on a standardized 0-4 ocular discomfort scale (0=no discomfort, 4=worst discomfort), and was recorded by study staff. When an eye manifested a score of greater than or equal to 3 at two consecutive assessments, 1-2 drops of the artificial tear was instilled in one eye, and 1-2 drops of the NSAID/artificial tear formulation in the contralateral eye, in a randomized fashion. Subjects recorded comfort of the drop immediately following instillation of the drop on a 0-9 comfort scale (0=extremely comfortable and 9=extremely uncomfortable) and remained in the CAE 60 more minutes, with ocular discomfort assessments every 5 minutes.

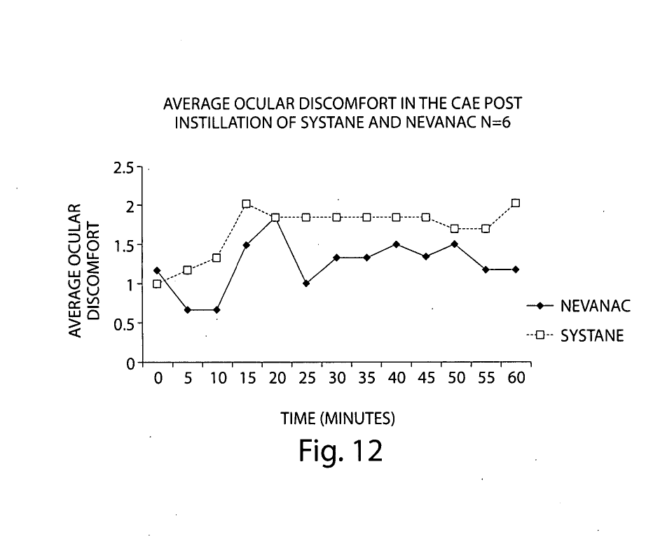

FIG. 12 depicts the results of this study. It was observed that the combined nepafenac/artificial tear formulation reduced ocular discomfort during the CAE challenge.

### REFERENCES

All publications and patents mentioned herein are hereby incorporated by reference in their entireties as if each individual publication or patent was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the invention described herein. While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations. Such equivalents are intended to be encompassed by the following claims.

We claim:

1. An ophthalmic formulation comprising a combination of:
1) a tear substitute component; and
2) a low-dose amount of an NSAID selected from the group consisting of ketorolac tromethamine, nepafenac, and bromfenac,
wherein the combination is effective to treat or prevent the signs and symptoms dry eye.

2. An ophthalmic formulation comprising a combination of:
1) a tear substitute component; and
2) a low dose amount of an NSAID selected from the group consisting of ketorolac tromethamine, nepafenac, and bromfenac,
wherein the low dose is an amount effective to reduce ocular surface discomfort without producing an anesthetic effect.

3. The ophthalmic formulation of claim 3, comprising about 0.15% to about 0.26% ketorolac tromethamine.

4. The ophthalmic formulation of claim 1, comprising about 0.03% to about 0.08% nepafenac.

5. The ophthalmic formulation of claim 5, comprising about 0.05% to about 0.065% nepafenac.

6. The ophthalmic formulation of claim 1, comprising about 0.027% to about 0.072% bromfenac.

7. The ophthalmic formulation of claim 7, comprising about 0.036% to about 0.059% bromfenac.

8. The ophthalmic formulation of claim 1, wherein the tear substitute component has a viscosity ranging from about 50-120 cpi.

9. The ophthalmic formulation of claim 8, wherein the tear substitute component has a viscosity ranging from about 70-120 cpi.

10. The ophthalmic formulation of claim 1, wherein the tear substitute component comprises an ingredient selected from the group consisting of: a polyol, a dextran, a water soluble protein, a carbomer, a gum, and a cellulose derivative.

11. The ophthalmic formulation of claim 10, wherein the cellulose derivative is selected from the group consisting of: hydroxypropylmethyl cellulose, carboxymethyl cellulose sodium, hydroxypropyl cellulose, hydroxyethyl cellulose, methyl cellulose, and one or more combinations thereof.

12. The ophthalmic formulation of claim 11, wherein the cellulose derivative is hydroxypropylmethyl cellulose (HPMC).

13. The ophthalmic formulation of claim 11, wherein the cellulose derivative is carboxymethyl cellulose sodium (CMC).

14. The ophthalmic formulation of claim 11, wherein cellulose derivative is a combination of CMC and HPMC.

15. An ophthalmic formulation comprising a combination of:
1) an HPMC-based tear substitute having a viscosity of about 50 centipoise, and
2) 0.2% ketorolac tromethamine,
wherein the combination is effective to treat or prevent the signs and symptoms dry eye.

16. An ophthalmic formulation comprising a combination of:
1) an HPMC-based tear substitute having a viscosity of about 50 centipoise, and
2) 0.05% nepafenac,
wherein the combination is effective to treat or prevent the signs and symptoms dry eye.

17. An ophthalmic formulation comprising a combination of:
1) an HPMC-based tear substitute having a viscosity of about 50 centipoise, and
2) 0.045% bromfenac,
wherein the combination is effective to treat or prevent the signs and symptoms dry eye.

18. An ophthalmic formulation comprising a combination of:
1) an HPMC-based tear substitute having a viscosity of about 70 centipoise, and
2) 0.25% ketorolac tromethamine,
wherein the combination is effective to treat or prevent the signs and symptoms dry eye.

19. A method of treating, and evaluating the treatment of, a subject having dry eye and/or eye irritation, comprising:
(a) determining a first measurement of the tear film break-up time (TFBUT) or ocular protection index (OPI) or non-invasive tear film break-up time in a subject and evaluating the patient's ocular discomfort;
(b) administering an ophthalmic formulation according to claim 1;
(c) determining a second measurement of the TFBUT or OPI or non-invasive tear film break up time in the subject;
wherein and an increase in the second measurement of TFBUT or OPI or non-invasive tear film break up time as compared to the first measurement indicates that the ophthalmic formulation is efficacious in treating the subject.
